(19) United States
(12) Patent Application Publication
(10) Pub. No.: US 2005/0220809 A1
(43) Pub. Date: Oct. 6, 2005

(54) Nucleotide sequence coding for a tolc and a defined amino acid sequence

(76) Inventors: Werner Goebel, GERBRUNN (DE); Ivaylo Genschev, Kist (DE); Simone Spreng, Bern (CH)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/505,620

(22) PCT Filed: Feb. 13, 2003
(86) PCT No.: PCT/DE03/00469

§ 371 Date: Mar. 30, 2005

(30) Foreign Application Priority Data
Feb. 20, 2002 (DE) 102 08 175.1

(51) Int. Cl. G01N 33/554; G01N 33/569; C07H 21/04; A61K 39/02; C12N 1/21; C07K 14/245

(52) U.S. Cl. 424/190.1; 536/23.7; 530/350; 435/252.33; 435/488; 435/7.32

(57) ABSTRACT

The invention relates to a nucleotide sequence coding for a TolC and a defined amino acid sequence, said defined amino acid sequence being inserted in the permissive, membrane-external area of the TolC, and several uses thereof, particularly bacteria containing such a nucleotide sequence.

### FIELD OF THE INVENTION

The invention relates to a nucleotide sequence coding for a TolC, a plasmid containing such a nucleotide sequence, a protein or a peptide coded by such a nucleotide sequence, a bacterium containing such a nucleotide sequence and several uses of such bacteria.

### BACKGROUND OF THE INVENTION AND PRIOR ART

Virulence-attenuated, intracellularly settling bacteria can induce a long-lasting immunity as live vaccines. Up to now, in particular Salmonella typhi TY1a (Levine et al., Lancet 1:1049-1052, 1987), Mycobacterium bovis BCG (Fine and Rodrigues, Lancet 335:1016-1020, 1990) and Vibrio cholerae (Levine and Kaper, Vaccine 11: 207-212, 1993) were used as live vaccines.

For instance, such variants of Listeria monocytogenes, Salmonella enterica sv. typhimurium and typhi, and BCG were already used as well-tolerated live vaccines against typhus and tuberculosis. These bacteria, including their attenuated mutants are generally immune-stimulating and can initiate a fair cellular immune response, and were therefore used a vaccine carriers.

The advantage of these bacteria as vaccine carriers is that they mainly induce a so-called Th1 immune response (Hess and Kaufmann, FEMS Immunol. Med. Microbiol. 23:165-173, 1999). This immune response is characterized by cytotoxic lymphocytes (CTL) and by the presence of specific IFN-gamma-secreting CD4+ T cells (also T helper cells, Th) (Abbas et al., Nature 383:787-793, 1996).

For instance, L. monocytogenes stimulates to a special extent via the activation of TH1-cells the proliferation of cytotoxic T lymphocytes (CTL). These bacteria supply secerned antigens directly into the cytosol of antigen-presenting cells (APC; macrophages and dendritic cells), which in turn express the co-stimulating molecules and cause an efficient stimulation of T cells. The listeriae are in part degraded in phagosomal compartments, and the antigens produced by these carrier bacteria can therefore on the one hand be presented by MHC class II molecules and thus lead to the induction of T helper cells. On the other hand, the listeriae replicate after release from the phagosome in the cytosol of APCs; antigens produced and secerned by these bacteria are therefore preferably presented via the MHC class I pathway, thus CTL responses against these antigens being induced. Further it could be shown that by the interaction of the listeriae with macrophages, natural killer cells (NK) and neutrophilic granulocytes, the expression of such cytokines (TNF-alpha, IFN-gamma, Il-2, IL-12; Unanue, Curr. Opin. Immunol. 9:35-43, 1997; Mata and Paterson, J. Immunol. 163:1449-14456, 1999) is induced, for which an antitumoral effectiveness was detected.

Recombinant bacteria were thus capable to protect against a heterologous tumor (Medina et al., Eur. J. Immunol. 29:693-699, 1999; Pan et al., Cancer Res. 59:5264-5269, 1999; Woodlock et al., J. Immunother. 22:251-259, 1999; Paglia et al., Blood 92:3172-3176, 1998; Paglia et al., Eur. J. Immunol. 27:1570-1575, 1997; Pan et al., Nat. Med. 1:471-477, 1995; Pan et al., Cancer Res. 55:4776-4779, 1995).

By the administration of L. monocytogenes, which were transduced for the expression of tumor antigens, the growth of experimental tumors could be inhibited antigen-specifically (Pan et al., Nat. Med. 1:471-477, 1995; Cancer Res. 59:5264-5269, 1999; Voest et al., Natl. Cancer Inst. 87:581-586, 1995; Beatty and Paterson, J. Immunol. 165:5502-5508, 2000).

Virulence-attenuated Salmonella enterica strains, into which nucleotide sequences coding for tumor antigens had been introduced, as tumor antigen-expressing bacterial carriers, could provide after oral administration a specific protection against different experimental tumors (Medina et al., Eur. J. Immunol. 30:768-777, 2000; Zoller and Christ, J. Immunol. 166:3440-34450, 2001; Xiang et al., PNAS 97:5492-5497, 2000).

Recombinant Salmonella strains were also effective as prophylactic vaccines against virus infections (HPV; Benyacoub et al., Infect. Immun. 67:3674-3679, 1999) and for the therapeutic treatment of a mouse tumor immortalized by a tumor virus (HPV) (Revaz et al., Virology 279:354-360, 2001).

For the use as a vaccine carrier, methods were developed to express expression products of nucleic acid sequences introduced into bacteria on the cell membrane of these bacteria or to have them secreted by these bacteria. The basis of these methods is the Escherichia coli hemolysin system hlyAs representing the prototype of a type I secretion system of gram-negative bacteria. By means of the hlyAs, secretion vectors were developed that allow an efficient discharge of protein antigens in Salmonella enterica, Yersinia enterocolitica and Vibrio cholerae. Such secretion vectors contain the cDNA of an arbitrary protein antigen coupled to the nucleotide sequence for the hlyA signal peptide, for the hemolysin secretion apparatus, hlyB and hlyD and the hly-specific promoter. By means of this secretion vector, a protein can for instance be expressed on the surface of this bacterium. Such genetically modified bacteria induce as vaccines a considerably stronger immune protection than bacteria, wherein the protein expressed by the introduced nucleic acid remains inside the cell (Donner et al., EP 1015023 A; Gentschev et al., Gene 179:133-140, 1996; Vaccine 19; 2621-2618, 2001; Hess et al., PNAS 93:1458-1463, 1996). The disadvantage of this system is however that by using the hly-specific promoter the amount of the protein expressed by the bacterium is small.

Further transport systems in bacteria represent for instance, i) the transport signal for the S-layer protein (Rsa A) of Caulobacter crescentus, where—for the secretion and the membrane-bound expression—the C-terminal RsaA transport signal is to be used (Umelo-Njaka et al., Vaccine 19:1406-1415, 20101), and ii) the transport signal for the Internalin A of Listeria monocytogenes. For the secretion, the N-terminal transport signal is necessary, and for the membrane-bound expression, the N-terminal transport signal together with the C-terminal part containing the LPXTG motive responsible for the cell wall anchoring (Dhar et al., Biochemistry 39:3725—3733, 2000).

In another context, the integral membrane protein TolC of E. coli is known. This is a multi-functional pore-forming protein of the outer membrane of E. coli, which, in addition to functions, such as e.g. reception of Colicin E1 (Morona et al., J. Bacteriol. 153:693-699, 1983) and the secretion of Colicin V (Fath et al., J. Bacteriol. 173:7549-7556, 1991), also serves as a receptor for the U3 phage (Austin et al., J. Bacteriol. 172:5312-5325, 1990). This protein is not only found in E. coli, but also in a multitude of gram-negative bacteria (Wiener, Structure Fold. Des. 8:171-5, 2000).

The crystal structure of the TolC protein shows that it forms, as a homotrimer, a tunnel channel having a length of about 120 Angstroms, the biggest part of the homodimer, the tunnel domain, being localized in the periplasm and only two little loops (amino acids 52-61 and 257-279) being settled on the surface of the bacterium (Koronakis et al., Nature 405:914-919, 2000). The tolC gene has the nucleotide sequence published by Niki et al., Nucleotide sequence of the tolC gene of Escherichia coli, Nucleic Acids Res. 18 (18), 5547 (1990). TolC is part of at least four different bacterial export systems by representing the membrane tunnel, through which the export of the bacterial protein is made possible. For instance in the hlyA transport system, the connection between hlyD and the periplasmic end of the TolC permits the export of the hemolysin from the hlyD into the membrane tunnel of the TolC (Gentschev et al., Trends in Microbiology 10:39-45, 2002).

### TECHNICAL OBJECT OF THE INVENTION

The invention is based on the technical object to specify a transport system, by means of which an expression product having a higher efficiency can be presented on an outer cell membrane.

### BASIC CONCEPT OF THE INVENTION AND PREFERRED EMBODIMENTS

For achieving the above technical object, the invention teaches a nucleotide sequence coding for a TolC and a defined amino acid sequence, said defined amino acid sequence being inserted in the permissive, membrane-external area of the TolC.

By the invention, a new transport system in gram-negative bacteria is achieved, by means of which larger amounts of a protein expressed by a gene within a bacterium can be transported on the outer cell membrane of the bacterium than was possible with prior art transport systems. Surprisingly, the transport system for the TolC protein of Escherichia coli permits a substantially stronger membrane-bound expression of a peptide or protein (arbitrary) than was known from prior art transport proteins, and that for a multitude of gram-negative bacteria. The membrane-bound expression of the defined amino acid sequence or gene product is exclusively achieved by the TolC.

A defined amino acid sequence may be an arbitrary given peptide or a protein, an arbitrary pharmaceutical active substance, an arbitrary antigen, an arbitrary antibody, or an arbitrary ligand.

The TolC may be a (wild-type) TolC protein according to ACCESSION X54049, to which hereby explicitly reference is made, or a (preferably N-terminal) partial sequence thereof or a mutant of the protein or of the partial sequence, for the partial sequence or the mutant the transport functionality being maintained. N-terminal partial sequences means any partial sequence beginning in the N-terminal area amino acids 1 to 50 of the TolC protein and ending at the C-terminal end of a loop, which is settled on the surface of the bacterium. Preferred is thus the N-terminal transport signal of TolC, but also the central part of the protein, which represents the extracellular areas of TolC. A mutant may comprise an insertion, deletion or substitution, as long as the transport functionality is not distinctly reduced thereby.

For certain applications, it may be recommended that a defined amino acid sequence be inserted on one side or both sides by a spacer sequence. This will however only be helpful, if the defined amino acid sequence is to be presented in a certain spatial structure, for instance in the case of an antigen, and this does however not take place by the defined amino acid sequence itself for steric or configurative reasons to a desired extent. Then a spacer sequence may be formed in particular by a sequence naturally following the defined amino acid sequence, thus the defined amino acid sequence being folded in the same way as in the natural antigen. The spacer sequence may however also be artificial, if thus a desired presentation and/or folding of the defined amino acid sequence is obtained. This can easily be calculated by means of theoretical methods, under consideration of the spatial conditions at the position of insertion in the TolC.

In detail it is preferred that the defined amino acid sequence is inserted in the N-terminal area of the TolC, in particular in the area of the amino acids 52 to 61 and/or 257 to 279 (each referred to the TolC protein).

Subject matter of the invention is further a plasmid containing a nucleotide sequence according to the invention and a protein or peptide coded by a nucleotide sequence according to the invention.

The invention further teaches a bacterium containing a nucleotide sequence according to the invention, the TolC causing the transport of the defined amino acid sequence on the membrane of the bacterium. In other words, in the bacterium is caused the membrane-bound expression of a gene product by the TolC protein. Subject matter of the invention is thus also a gram-negative bacterium, which contains at least one nucleotide sequence coding for at least one defined amino acid sequence and for at least one E. coli TolC gene product. This E. coli TolC gene product preferably is wild-type. Subject matter of the invention are however also mutated E. coli TolC gene products, wherein the transport signal activity has been maintained. Preferably, the bacterium is selected from the group composed of “Salmonella spp., Escherichia coli, Vibrio cholerae, Pseudomonas aeruginosa, Shigella spp. and Yersinia spp.”.

Nucleotide sequences and bacteria can be used for various applications. For instance, the invention also teaches a pharmaceutical composition containing a bacterium according to the invention, and as an option at least one physiologically tolerable carrier substance, wherein the defined amino acid sequence is selected according to a given substance to be bound in an organism. By means of such a pharmaceutical composition, substances interfering with the normal cellular metabolism, for instance exogenous toxicants or mutation-caused endogenous substances such as octagons can be bound and thus inhibited. Further, by binding certain cellular target substances, metabolism processes can be modulated by removal of normal complex partners or those being regulated-up because of a disease. Thereby, for instance a defined association is inhibited, and the shuttle related thereto is regulated down. Such a process can in turn be used for regulating-up other related processes. Insofar, the defined amino acid sequence needs only be selected according to the target molecule to be inhibited with high specificity. Such a pharmaceutical composition thus serves at last for therapeutic purposes.

A pharmaceutical composition suitable for vaccination purposes contains a bacterium according to the invention and as an option at least one physiologically tolerable carrier substance, wherein the defined amino acid sequence is an immunization sequence. An immunization sequence stimulates in an organism the generation of antibodies against a natural antigen, which contains as a partial sequence the immunization sequence or is composed thereof.

For diagnostic purposes, the invention teaches a diagnostic kit containing a bacterium according to one of claims 7 to 9, wherein the defined amino acid sequence specifically binds a marker substance to be determined. If for instance a tissue or fluid sample is taken from an organism, and this sample, if applicable after a pre-treatment with separation of undesired sample components, is incubated with the bacterium, the binding events at the defined amino acid sequence can be detected, and in case of a binding event, it is detected that the substance specifically binding to the defined amino acid sequence is contained in the sample. The detection of binding events can be made in various ways well known to the average man skilled in the art.

Finally, the invention teaches a preparative binding substance containing a bacterium according to the invention, wherein the defined amino acid sequence specifically binds a target substance to be separated from a solution. By such a binding substance, undesired substances can on the one hand specifically be removed from a solution by that the solution is incubated with the bacterium, and the bacterium is discarded after separation. On the other hand, a separation or an enrichment of a target substance may be performed in a corresponding manner, namely by that after the incubation the target substance is eluted from the bacterium. In this context, too, the invention can be used for the separation and/or enrichment of antigens, of antibodies, peptides, proteins or ligands.

### EXAMPLES OF EXECUTION

### Example 1

### Preparation of the TolC Vector

The tolC gene of E. coli including its wild-type promoter was amplified by means of PCR (1 min 94° C., 1 min 66° C., 1 min 30 s 72° C.) with the oligonucleotides 5′TolC (5′-TAACGCCCTATGTCGACTAACGCCAACCTT-3′) and 3′TolC (5′-AGAGGATGTCGACTCGAAATTGAAGCGAGA-3′) from the plasmid pAX629 (C. Wandersman, Institute Pasteur, Paris). At both ends, an additional SalI interface was introduced. The purified PCR product (QIAquick PCR Purification Kit—Qiagen, Hilden, Germany) was digested with the restriction endonuclease SalI and cloned into the vector pBR322 pre-split with SalI. The vector thus constructed was designated pBR322 tolC. The functionality of the cloned tolC gene was then investigated in several tests.

### Example 2

### Introduction of an Antigen Sequence into the Sequence of the TolC Protein

In the tolC sequence coding for one of the extracellular loops, a KpnI interface was identified. This was used for cloning antigenic peptide sequences of the p60 protein (iap gene) of Listeria monocytogenes and permitted an insertion of foreign antigens behind amino acid 271 of the mature TolC protein.

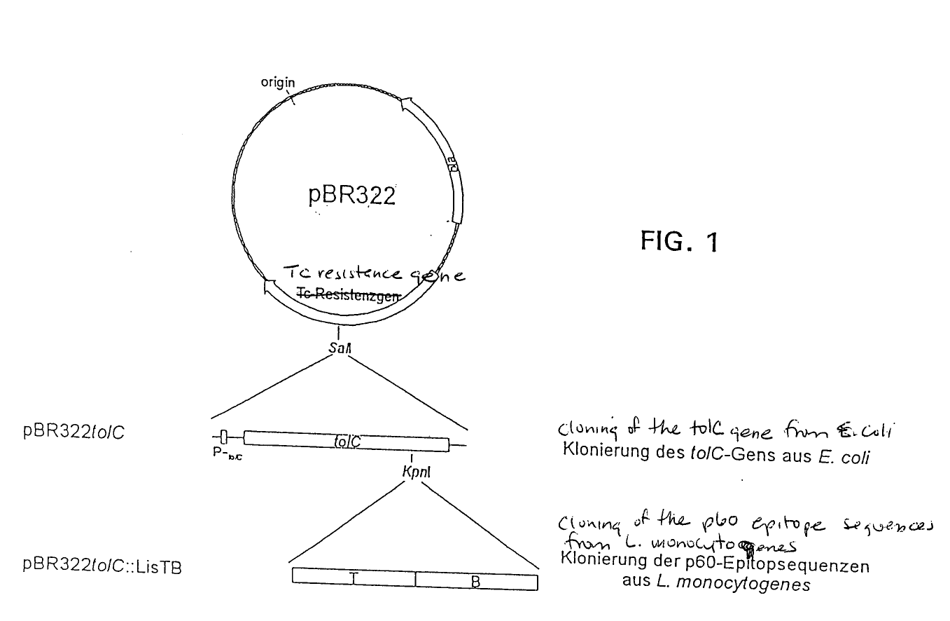

The iap sequence coding for a B cell epitope (amino acids 291-301) and a CD4-restringed T cell epitope (amino acids 301-312) of the p60 protein was cloned as a KpnI fragment into the vector pBR322 tolC pre-cut with KpnI (FIG. 1). The plasmid thus obtained was designated pBR322-tolC::LisTB.

FIG. 1 shows the cloning strategy for the insertion of the p60-specific epitope sequences into the wild-type plasmid-coded E. coli tolC gene on the vector pBR322. There are: bla—ampicillin resistance gene; Tc—tetracycline; T—L. monocytogenes p60 T cell epitope (AS 301-312); B—L. monocytogenes p60 B cell epitope (AS 291-301); PtolC—wild-type E. coli tolC promoter.

### Example 3

### Expression of the Antigen on the Membrane of a Gram-Negative Bacterium (Escherichia coli)

The expression of the epitopes of the p60 protein from L. monocytogenes within the TolC protein was detected in a Western blot. For this purpose, cell lysate proteins of E. coli CC118 tolC, E. coli CC118tolC/pBR322tolC and E. coli CC118tolC/pBR322tolC::ListTB were isolated in the late logarithmic phase. The applied cell protein totals corresponded to approx. 100 millions bacteria. The proteins were separated in a 15% SDS polyacrylamide gel, and the expression of the chimeric TolC proteins or of the inserted epitopes were detected on the one hand with a polyclonal serum against the TolC protein and on the other hand with the monoclonal antibody K317 (Rowan et al., J. Clin. Microbiol. 38:2643-2648, 2000) specifically directed against the B cell epitope from L. monocytogenes (FIG. 2B).

As expected, no TolC protein could be detected in the cell lysate of E. coli CC118tolC, which can be explained by a mutation in the chromosomal tolC gene in this strain (Schlor et al., Mol. Gen. Genet. 256:306-319, 1997). The complementation with pBR322tolC led in this strain to the expression of the 52 kDa large TolC protein. The insertion of the L. monocytogenes epitopes into the TolC protein did not affect the expression of TolC and led to a slight modification of the size of the chimeric protein of approx. 3 kDa.

The expression of the p60-specific epitopes in E. coli CC118tolC/pBR322tolC::ListB could be confirmed with the monoclonal p60 antibody K317.

### Example 4

### Detection of the Exposed Localization of the L. monocytogenes p60 Epitopes in Salmonella enteritidis SM6T (tolC)

Since the insertion position of the two listerial p60 epitopes was in an extracellular loop of TolC behind amino acid 271 of the mature protein, they should be present in an exposed manner at the surface of S. enteritidis SM6T (Stone et al., Mol. Microbiol. 17:701-712, 1995). The definitive extracellular localization of the p60-specific epitopes in S. enteritidis SM6T was tested by indirect immunofluorescence. 25 μl each of an overnight culture of S. enteritidis SM6T/pBR322tolC and S. enteritidis SM6T/pBR322 tolC::LisTB were dropped onto object carriers and air-dried. The cells were stained with the monoclonal p60 antibody K317 (1:200), and bound antibodies were then detected with an FITC-labeled secondary anti-mouse serum (Dianova, Germany, working titer: 1:40).

The fluorescence-microscopic analysis confirmed the extracellular localization of the L. monocytogenes-specific epitopes in the strain S. enteritidis SM6T/pBR322tolC::LisTB.

### Example 5

### Immunization Tests with the Gram-Negative Bacterium and Analysis of the Protective Immune Responses after Infection with Wild-Type L. monocytogenes

In order to find out whether the exposed expression of the T cell epitope from the p60 protein of L. monocytogenes in the murine listeriosis model leads to a protection, 8 female balb/c mice (Charles River, Sulzfeld, Germany) having an age of six weeks were orally immunized with a dose of 1×10⁷ S. enteritidis SM6T/pBR322tolC::LisTB. For control purposes, 5 female mice were orally immunized with S. enteritidis SM6T. The animals were immunized a second time three weeks later with the same dose of bacteria.

The immunization success was tested five weeks after the first immunization in an immune blot, on which were applied supernatant proteins of Listeria monocytogenes. Anti-p60-specific antibodies could be detected in the serum of the mice immunized with S. enteritidis SM6T/pBR322-tolC::LisTB.

Three weeks after the second immunization, the animals were intravenously infected with 5×10⁴ L. monocytogenes EGD, the five-fold LD50. Whilst the survival rate for the balb/c mice immunized with S. enteritidis SM6T/pBR322tolC::LisTB after intravenous infection with L. monocytogenes EGD was 88%, the survival rate in the control group was only 20%.

Thus the expression of the p60-specific epitopes within an extracellular loop of TolC in the attenuated S. enteritidis carrier strain SM6T led to the induction of Listeria monocytogenes-specific immune responses, which were capable to protect balb/c mice against a usually lethal infection. Since the induction of antibodies against the B cell epitope from the p60 protein could be detected in a Western blot, it is obvious that an immune reaction under participation of the antibodies has caused the observed protection of the mice against an otherwise lethal infection with L. monocytogenes.

1. A nucleotide sequence coding for a TolC and a defined amino acid sequence, wherein the defined amino acid sequence is inserted in the permissive, membrane-external area of the TolC.

2. A nucleotide sequence according to claim 1, wherein the TolC is a TolC protein according to ACCESSION X54049 or a preferably N-terminal partial sequence thereof or a mutant of the protein or of the partial sequence, and wherein for the N-terminal partial sequence or the mutant the transport functionality is maintained.

3. A nucleotide sequence according to claim 1, wherein the defined amino acid sequence is inserted on one side or both sides by a spacer sequence.

4. A nucleotide sequence according to claim 1, wherein the defined amino acid sequence is inserted in the N-terminal area of the TolC, in particular in the area of the amino acids 52 to 61 and/or 257 to 279 (each referred to the TolC protein.

5. A plasmid containing a nucleotide sequence according to one of claims 1 to 4.

6. A protein or a peptide coded by a nucleotide sequence according to one of claims 1 to 4.

7. A bacterium containing nucleotide sequence according to claim 1, wherein the TolC causes the transport of the defined amino acid sequence on the membrane of the bacterium.

8. A bacterium according to claim 7, wherein the defined amino acid sequence represents a peptide, a protein, an active substance, an antigen, an antibody or a ligand.

9. A bacterium according to claim 7, selected from the group composed of “Salmonella spp., Escherichia coli, Vibrio cholerae, Pseudomonas aeruginosa, Shigella spp and Yersinia spp.”

10. A pharmaceutical composition containing a bacterium according to claim 7, and at least one physiologically tolerable carrier substance, wherein the defined amino acid sequence is selected according to a given substance to be bound in an organism.

11. A pharmaceutical composition containing a bacterium according to claim 7 and at least one physiologically tolerable carrier substance, wherein the defined amino acid sequence is an immunization sequence.

12. A diagnostic kit containing a bacterium according to claim 7, wherein the defined amino acid sequence specifically binds a marker substance to be determined.

13. A preparative binding substance containing a bacterium according to claim 7, wherein the defined amino acid sequence specifically binds a target substance to be separated from a solution.

14. A nucleotide sequence according to claim 2, wherein the defined amino acid sequence is inserted on one side or both sides by a spacer sequence.

15. A nucleotide sequence according to claim 2 wherein the defined amino acid sequence is inserted in the N-terminal area of the TolC, in particular in the area of the amino acids 52 to 61 and/or 257 to 279 (each referred to the TolC protein.
